(19) United States
(12) Patent Application Publication
(10) Pub. No.: US 2019/0152024 A1
(43) Pub. Date: May 23, 2019

(54) Stripop

(71) Applicant: Jacob Lee Downey, I, Cordova, IL (US)

(72) Inventor: Jacob Lee Downey, I, Cordova, IL (US)

(21) Appl. No.: 15/817,231

(22) Filed: Nov. 19, 2017

(51) Int. Cl. B25B 9/02; A61F 15/00

(52) U.S. Cl. CPC B25B 9/02; A61F 15/002

(57) ABSTRACT

Stripop is a device designed to split, cleave, or separate bandage packaging, bags, and/or protective backing or layering. Stripop applies adhesive surfaces, and/or gripping mechanisms, and a spring, manual, or electronic action; to grasp, then split, cleave, or separate a bandage package, bag, or protective backing or layering.

### SUMMARY OF THE INVENTION

### Field of Invention

The present invention relates to the field of health care & health assistance. Similar to a “pill crusher”, or “bandage dispenser”; this device will help those who are providing health care & health assistance.

### Background of the Invention

Stripop was developed to open a bandage package; by grasping the opposing sides of the package, then splitting them apart. Ideally the split will cleave along the packages adhesion line.

The idea for the technology came to me while attempting to think of a technology that would help people.

I thought of how nice it would be to easily open a bandage package, then pondered ways to achieve that; using simple economically friendly technology.

### SUMMARY OF THE INVENTION

Stripop is a pair of tongs with two sticky surfaces. The sticky surfaces are attached to the tongs on opposing tines. The tongs have enough spring power to split a bandage package.

When attempting to open a bandage package, the user will place the bandage package between the two sticky surfaces, then press the tong's tines together; until the sticky surfaces have made contact with the opposing sides of the bandage package. After which the user can quickly release the tongs to split the package open.

Alternatively, stripop may also be made with two gripping mechanisms, or a combination of sticky and gripping mechanisms; and may also be used to cleave bags, and separate protective layering; for example: One gripping tine could grasp a portion of adhesive backed Velcro, while the sticky tine grasps the protective backing, and then separates the two.

### DESCRIPTION OF DRAWINGS

FIG. 1: Is an axonometric perspective of one possible configuration of the invention; using adhesive pads; with a bandage package split open; as embodied by the current prototype. And is an example of the invention in use.

FIG. 2: Is an axonometric perspective of one possible configuration of the invention; using one adhesive pad, and one mechanical gripping mechanism; with an adhesive backed loop strip from a hook and loop fastener assembly; being separated from its protective backing, included; as embodied by a possible version.

FIG. 3: Is a side perspective of the parts explosion; containing the two primary components; the tong, and the adhesive pad and/or gripping mechanism; and the third optional component; the spring; used in the current prototype; as displayed in FIG. 1.

FIG. 4A. Is a side perspective of one possible pair of tongs that could be used; and is embodied by the current prototype.

FIG. 4B. Is a side perspective of a pair one possible type of adhesion pad that could be used; and is embodied by the current prototype.

FIG. 4C. Is a side perspective of one possible spring that could be used; and is embodied by the current prototype.

### DETAILED DESCRIPTION OF THE EMBODIMENTS

Index:
Section 1: Main description
Section 2: Construction
Section 3: Operation
Section 4: Properties

Section 1: Main Description

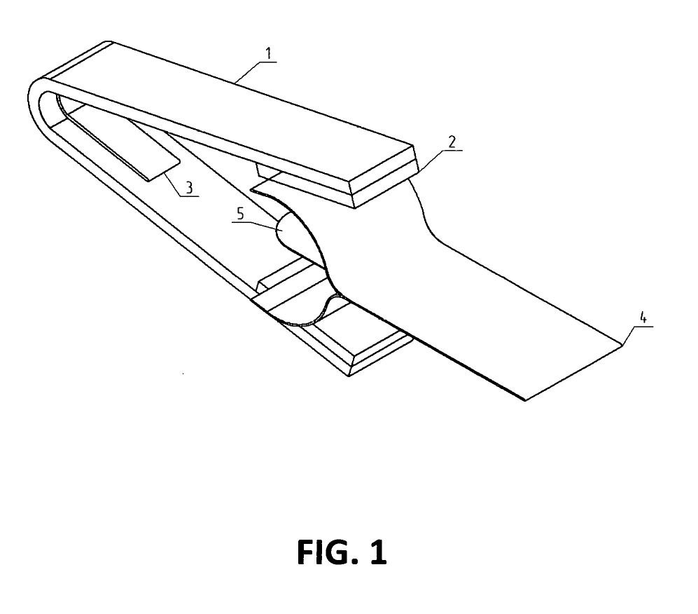

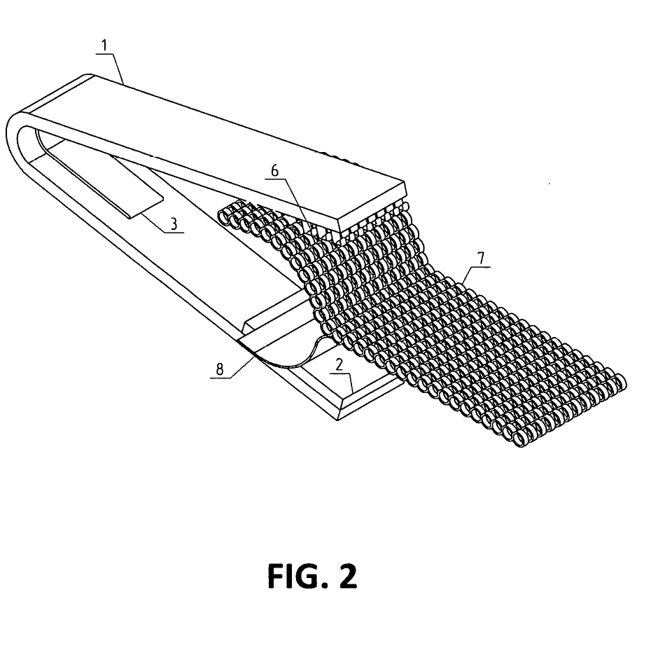

Stripop is a pair of tongs with adhesive and/or gripping surfaces; such as in claim 1-3; an example of which is shown in FIG. 1 & FIG. 2.

The adhesive surfaces, and/or gripping mechanisms, are attached to the tongs on opposing tines.

The tongs may or may not have enough spring power included, or incorporated, to operate. The tongs may or may not require manual power to operate. The tongs may or may not include electronic power; included or incorporated; to operate.

Section 2: Construction

(2a) What it Looks Like.

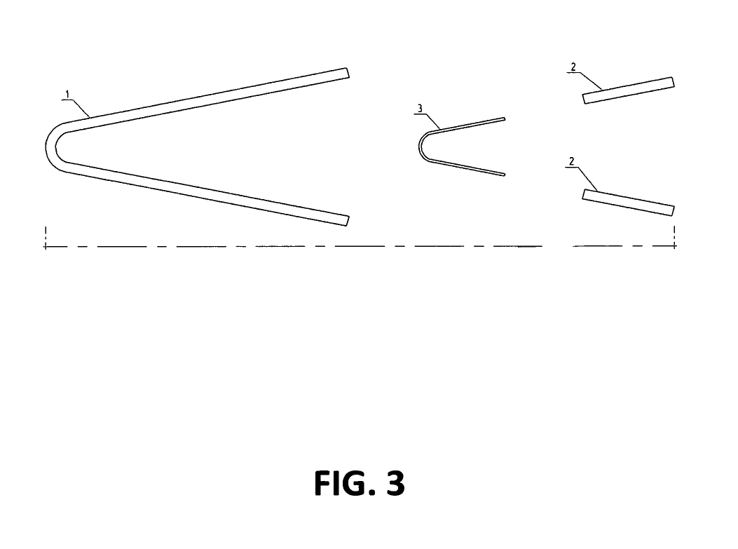

Stripop resembles a pair of tongs (FIG. 1-3, reference numeral 1). The tweezers or pincers portion of the tongs have adhesive pads (FIGS. 1,2, & 4B, reference numeral 2) and/or gripping mechanisms (FIG. 2, reference numeral 6), designed to adhere to or grip substrates; which the user wants to split, cleave, or separate; such as a bandage package (FIG. 1, reference numeral 5) containing a bandage (FIG. 1, reference numeral 4), or adhesive backing or layering; such as adhesive backed hook and loop fastener; where the mechanical portion might provide the hook portion (FIG. 2, reference numeral 6), to separate an adhesive backed loop strip (FIG. 1, reference numeral 7), from its protective backing (FIG. 1, reference numeral 8).

(2b) How to Make it.

The following describes the requirements used to embody the technology's desired goal.

Stripop should be constructed of tongs; either spring operated, manually operated, electronically operated, or any combination thereof.

The tong (meeting) point of the tines should incorporate an adhesive surface, gripping mechanism, or any combination thereof.

The current embodiment of the technology, (claim 1) (FIG. 1, reference numeral 1-3), is typically constructed of 4 parts.

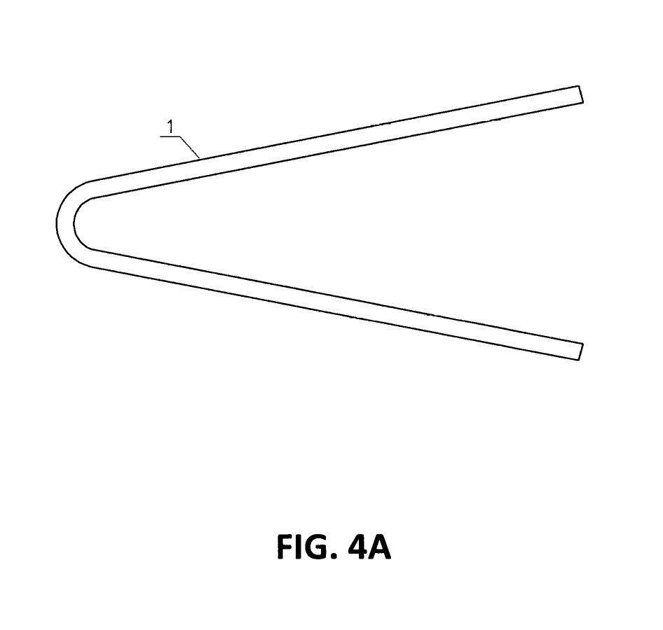

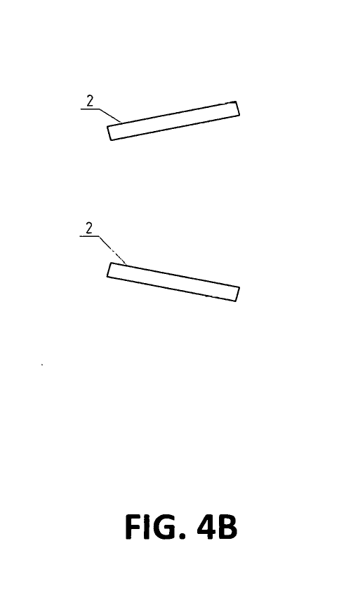

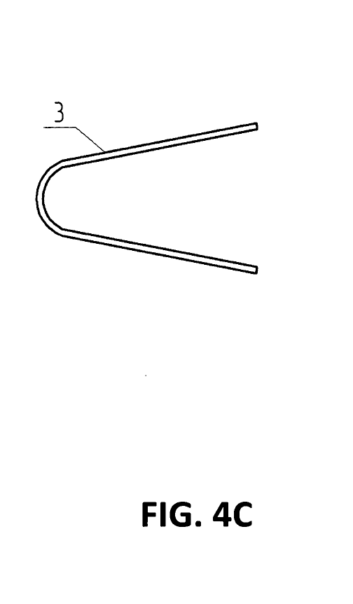

Part 1) A pair of tongs (FIG. 4A, reference numeral 1).

Part 2 & 3) Two adhesive pads (FIG. 4B, reference numeral 2).

Part 4) A spring (FIG. 4B, reference numeral 3)

Part 1) The tongs are meant to provide a meeting place for the adhesive pads, and/or gripper mechanisms (claim 1-3). The tongs may be any size or shape; provided it allows the adhesive pads or gripper mechanisms to meet.

Part 2 & 3) An adhesive substrate and/or gripper mechanism should be attached to the tongs tine tips at the meeting point (claim 1-3). Ideally the adhesive substrate will have an adhesion force greater than the force needed to split, cleave, or separate a bandage package, bag, or protective backing or layering (claim 7-9).

Part 4) As an option; the prototype has a spring incorporated in the tong (claims 4 & 5).

Ideally the spring will contain or add enough force to split, cleave, or separate a bandage package, bag, or protective backing or layering (claim 7-9).

Section 3: Operation

When attempting to open a bandage package, the user will place the bandage package between the two adhesive and/or gripping surfaces, and then press the tong's tines together; until the adhesive and/or gripping surfaces have adhered to or grasped the opposing sides of the bandage package. After which the user should quickly release the tongs; to split the package open.

When attempting to open a bag, the user will place the bag between the two adhesive and/or gripping surfaces, then press the tong's tines together; until the adhesive and/or gripping surfaces have adhered to or grasped the opposing sides of the bag. After which the user should quickly release the tongs; to cleave the bag open.

When attempting to separate a protective backing or layering, the user will place the protective backing or layering between the two adhesive and/or gripping surfaces, then press the tong's tines together; until the adhesive and/or gripping surfaces have adhered to or grasped the opposing sides of the protective backing or layering. After which the user should quickly release the tongs; to separate the protective backing or layering.

Section 4: Properties

When pressed together the opposing surfaces of the tongs will adhere to or grip the desired substrates.

When released the tongs; spring, manual, or electronic operation will apply force, through the adhesive or gripping surfaces, to split, cleave, or separate the desired substrates.

1. Tongs with opposing adhesive surfaces.

2. Tongs with opposing gripping surfaces; such as a pinch point, micro texture, friction inducing surface, or hook and loop fastener gripping end; possibly used to separate adhesive backing from Velcro.

3. Tongs with an opposing adhesive surface and gripping surface.

4. Tongs, as in claim 1-3; with a spring force included.

5. Tongs, as in claim 1-3; without spring; such as with manual operation, or electronic operation.

6. Tongs, as in claim 1-5; built into a package; such as the side of a box, or part of a clamshell package.

7. Tongs, as in claim 1-5; designed as, and or sold as, a bandage package opener.

8. Tongs, as in claim 1-5; designed as, and or sold as, a protective backing or layering separator; such as for stickers, window film, or adhesive backed hook and loop fastener.

9. Tongs, as in claim 1-5; designed as, and or sold as, a bag opener; such as for trash bags, shopping bags, or produce bags.
